(19) United States
(12) Patent Application Publication
(10) Pub. No.: US 2026/0021243 A1
(43) Pub. Date: Jan. 22, 2026

(54) LAVAGE SYSTEMS AND DEVICES HAVING A FLOW-ADJUSTING MEMBER

(71) Applicant: CareFusion 2200, Inc., San Diego, CA (US)

(72) Inventors: John Anthony KRUEGER, San Diego, CA (US); Christopher MCGINLEY, San Diego, CA (US); Kenneth Bruce THURMOND, San Diego, CA (US); Maleeha MASHIATULLA, San Diego, CA (US); Katya PEREZ, San Diego, CA (US); Michael PLISHKA, San Diego, CA (US)

(21) Appl. No.: 18/881,420

(22) PCT Filed: Jul. 5, 2023
(86) PCT No.: PCT/US2023/026928

§ 371 Date: Jan. 6, 2025

Related U.S. Application Data
(60) Provisional application No. 63/358,401, filed on Jul. 5, 2022.

(51) Int. Cl. A61M 3/02

(52) U.S. Cl. CPC A61M 3/0262; A61M 3/0279

(57) ABSTRACT

A system for applying a lavage fluid to a surface, the system having a sealed body for housing a lavage fluid, and a flow-adjusting member being configured to create a disruption in the sealed body dispense the lavage fluid, wherein the sealed body and lavage fluid contained therein are terminally-sterilized.

### CROSS REFERENCE TO RELATED APPLICATIONS

The present application claims priority to U.S. Provisional Application No. 63/358,401, filed on Jul. 5, 2022, the entire content of which is incorporated herein by reference.

### TECHNICAL FIELD

The present disclosure is directed to devices and systems for applying a lavage fluid to a surface, the devices and systems comprising at least one flow-adjusting member.

### BACKGROUND

Currently, lavage (that is, the washing out of a body cavity, surgical cavity, or external wound with a medically acceptable fluid) is often employed to prevent contamination of an open surgical wound, which may occur for a variety of reasons such as accidental visceral entry or perforated viscus, operations complicated by gross spillage, departure from sterile technique, and/or existing, ongoing clinical infection. Lavage processes are thus often employed to provide intraoperative antiseptic wound irrigation.

The art of lavage currently embraces a wide variety of different approaches that vary based on the situation (for example, the size and shape of the cavity or wound) and on the medical practitioner performing the lavage process (for example, a practitioner's technique preference). Currently, no specific lavage technique is standard in the art, and as such, medical facilities often require numerous different lavage devices and systems to accommodate the variety of potential approaches. The presentation of such devices and systems is also sometimes a concern, as the inadvertently inappropriate use of such devices and systems (e.g., intravenously, if the device and/or system has a similar appearance to an intravenous device and/or system) could results in devastating effects.

Moreover, several drawbacks exist with current lavage practices, including insufficiencies in antiseptic fluid properties (e.g., the amount of time necessary for an antiseptic fluid to achieve an acceptable biological effect, which may be prohibitive), the risk of systemic absorption of the antiseptic fluid, adverse reactions such as anaphylaxis, peritoneal adhesions, neurotoxicity, and respiratory insufficiency, and improper dosage or contamination of the antiseptic fluid, which are sometimes prepared ad hoc by a medical practitioner performing the lavage.

In addition, current lavage devices often pose an increased risk of contamination. For example, some current lavage devices utilize a resilient hollow body that expel lavage fluid upon pressure applied thereto (e.g., a “squeeze bottle” or the like). In order to function, such devices require one or more re-equilibrium periods (i.e., a period of time wherein a reduced or no pressure is applied to the device) such that a gas such as air may be pulled into the device sufficient to re-equilibrate its internal pressure. However, by introducing non-sterile gas into the device, the sterility of a lavage fluid contained therein (and thus, surgical wounds contacted by the same) may be jeopardized. Furthermore, such devices intake gas during re-equilibrium periods along the same path that lavage fluid is dispensed from the device. However, these paths are generally not optimized for such a function, and as such, the re-equilibrium periods required by such devices often provide an unacceptable delay to the lavage process, which often results in an inefficient and/or ineffective lavage process. Further, current lavage devices provide only limited ability to the clinician to control and target the flow of the lavage fluid

There is thus a need in the art for versatile devices and systems for performing lavage processes, and in particular, devices and systems that enable medical practitioners to safely and effectively reduce contamination in surgical wounds that are susceptible to surgical site infections. There is a need in the art for terminally sterilized lavage devices, wherein the lavage fluid contained therein is terminally sterilized along with the lavage devices, without damaging the materials used in production of the lavage devices. There is also a need in the art for lavage devices that have the ability to deliver significant amounts of lavage fluid in a short period of time, wherein the clinician can optimally control and target the flow of the lavage fluid.

### SUMMARY

The present disclosure is directed to devices and systems for delivering a lavage fluid, such as an irrigation solution, to a surface. The device comprises a sealed body that is configured to house a lavage fluid, such as an irrigation solution. Optionally, both the sealed body and the lavage fluid contained therein are terminally sterilized. The system further comprises at least one flow-adjusting member, wherein the at least one flow-adjusting member is configured to facilitate the process of applying the lavage fluid to a surface sufficient for a lavage process.

The devices and systems may be adaptable such that a user may select from one or more different fluid flow rates, fluid flow designs, and/or fluid flow forces, thus providing selectable control of lavage fluid delivery to a surface. The present disclosure is also directed to methods of using the devices and systems described herein.

The present disclosure is also directed to the flow-adjusting member useable with the devices and systems as described herein. The flow-adjusting member is configured to create a disruption in the sealed body containing the lavage fluid. The disruption created in the sealed body facilitates the discharge of the lavage fluid in a flow pattern from the sealed body. The flow-adjusting member may or may not be in fluid communication with the body during the discharge of lavage fluid.

### BRIEF DESCRIPTION OF THE DRAWINGS

FIG. 1 shows an example of a sealed body according to the aspects of the present disclosure.

FIG. 2 shows an example of a flow-adjusting member according to the aspects of the present disclosure.

FIG. 3 shows an example of a sealed body according to the aspects of the present disclosure.

FIG. 4 shows an example of a sealed body and a flow-adjusting member according to the aspects of the present disclosure.

FIG. 5 shows examples of flow-adjusting members according to the aspects of the present disclosure.

FIG. 6 shows an example of a flow-adjusting member according to the aspects of the present disclosure.

FIG. 7 shows an example of a flow-adjusting member and a sealed body according to the aspects of the present disclosure.

FIG. 8 shows an example of a flow-adjusting member according to the aspects of the present disclosure.

### DETAILED DESCRIPTION

The present disclosure is directed to devices and systems for delivering a lavage fluid, such as an irrigation solution, to a surface. The system comprises a device, such as a fluid receptacle, comprising a sealed body that is configured to house a lavage fluid, such as an irrigation solution. The system further comprises at least one flow-adjusting member, wherein the at least one flow-adjusting member is configured to facilitate the process of applying the lavage fluid to a surface sufficient for a lavage process. The devices and systems may be adaptable such that a user may select from one or more different fluid flow rates, fluid flow designs, and/or fluid flow forces, thus providing selectable control of lavage fluid delivery to a surface. The present disclosure is also directed to methods of using the devices and systems described herein.

The present disclosure is also directed to the flow-adjusting member useable with the devices and systems as described herein. The flow-adjusting member is configured to create a disruption, such as a cut, puncture, hole, or slit, in the sealed body configured to house the lavage fluid. The disruption created in the sealed body facilitates the discharge of the lavage fluid in a flow pattern from the sealed body. The flow-adjusting member may or may not be in fluid communication with the sealed body during the discharge of lavage fluid.

As used herein, the term “lavage fluid” refers to a fluid suitable for a lavage process as described herein. As used herein, “lavage” refers to the irrigation of a body cavity, a surgical cavity, and/or an external wound.

According to some aspects, the lavage fluid may comprise an irrigation solution. As used herein, an “irrigation solution” refers to a solution comprising at least a solvent and one or more chemical agents. Non-limiting examples of chemical agents include, but are not limited to, antiseptic agents, antibiotic agents, antimicrobial agents, oxidizing agents, and combinations thereof. According to some aspects, the irrigation solution is an aqueous solution. As used herein, the term “aqueous solution” refers to a solution wherein the solvent comprises at least a majority of water. It should be understood that in some examples, the solvent may consist of water. According to some aspects, the irrigation solution is an alcoholic solution. As used herein, the term “alcoholic solution” refers to a solution wherein the solvent comprises at least a majority of alcohol. It should be understood that in some examples, the solvent may consist of one or more alcohols. Non-limiting examples of alcohols include, but are not limited to, ethanol, isopropyl alcohol, n-propanol, and combinations thereof.

In one non-limiting example, the chemical agent may comprise an antiseptic agent. According to some aspects, the antiseptic agent may comprise a cationic molecule (i.e., a molecule having a positive charge), such as a cationic surfactant or a cationic biguanide derivative (i.e., a compound derived from biguanide). According to some aspects, the antiseptic agent may comprise a bis-(dihydropyridinyl)-decane derivative (i.e., a compound derived from bis-(dihydropyridinyl)-decane). According to some aspects, the antiseptic agent may comprise an octenidine salt and/or a chlorhexidine salt. According to some aspects, the antiseptic agent may comprise alexidine, octenidine dihydrochloride, chlorhexidine gluconate, or a combination thereof.

Additionally or alternatively, the antiseptic agent may comprise iodine. According to some aspects, the iodine may be provided as an iodine complex, such as povidone-iodine (PVPI), nonylphenoxy-(ethyleneoxy)-iodine, polyethylene oxy polypropleneoxy-iodine, undecoylinium-chloride-iodine, iodine povacrylex, and combinations thereof.

Additionally or alternatively, the chemical agent may comprise an oxidant (i.e., an oxidizing agent). Non-limiting examples of oxidants according to the present disclosure include, but are not limited to, sodium hypochlorite, hydrogen peroxide, Dakin's solution, hypochlorous acid, and combinations thereof.

In one non-limiting example, the chemical agent may comprise an antibiotic agent. Non-limiting examples of antibiotic agents according to the present disclosure include, but are not limited to, bacitracin, vancomycin, gentamycin, cefazolin, clindamycin, polymyxin, or a combination thereof.

The chemical agent may have an antimicrobial activity sufficient to provide an acceptable log reduction of microbes in a certain time period. It should be understood that as used herein, the term “microbes” may refer to any microorganism to be killed and/or removed as a result of lavage. Example microbes include bacteria, fungi, viruses, and combinations thereof.

Example bacteria include drug-resistant and drug-sensitive, but are not limited to, Streptococcus spp. (e.g., S. mutans, S. pyogenes, S. salivarius, S. sanguis), Staphylococcus spp. (e.g., S. aureus, S. epidermidis, S. haemolyticus, S. hominis, S. simulans, S. saprophyticus), Enterococcus spp. (e.g., E. faecalis E. faecium, and E. hirae), Bacteroides fragilis, Cutibacterium acnes (formerly Propionibacterium acnes), Clostridium difficile (spore and vegetative cells), Pseudomonas aeruginosa, Escherichia coli. Burkholderia cepacia, Proteus mirabilis, Klebsiella spp. (e.g., K. aerogenes, K. pneumoniae), Acinetobacter baumannii, Micrococus luteus, Haemophilus Influenza, and Serratia marcescens.

Example fungi include drug-resistant and drug-sensitive, but are not limited to, Aspergillus brasiliensis. Candida spp. (C. albicans, C. aurus, C. dubliniensis, C. glabrata, C. guillermondii, C. kefyr (formerly C. pseudotropicalis), C. krusei, C. lusitaniae, C. tropicalis), Epidermophyton floccosum. Microsporum spp (e.g., M. gypseum. M. canis), and Trichophyton mentagrophytes.

Example viruses include, but are not limited to, DNA and RNA genomes that are single stranded or double stranded, have sense or antisense orientation, protein coat (capsid) with or without a lipid envelope, such as, cytomegalovirus (CMV), human immunodeficiency virus (HIV), herpes simplex virus types 1 (HSV-1) and 2 (HSV-2), influenza virus, parainfluenza virus, norovirus, and coronavirus.

According to some aspects, the certain time period may be a period of no more than about five minutes, optionally no more than about four minutes, optionally no more than about three minutes, optionally no more than about two minutes, and optionally no more than about one minute.

According to some aspects, the certain time period may be no more than about 120 seconds, optionally no more than about 105 seconds, optionally no more than about 90 second, optionally no more than about 75 seconds, optionally no more than about 60 seconds, optionally no more than about 45 seconds, optionally no more than about 30 seconds, and optionally no more than about 15 seconds.

It should be understood that “an acceptable log reduction” may be microbe-dependent. For example, an acceptable log reduction as described herein may refer to an acceptable log reduction of one type of microbe present on a surface (e.g., present in a body cavity or at an external wound site), a combination of two more types of microbes present on a surface, or total microbes present on a surface.

According to some aspects, an acceptable log reduction may be at least about 1.0, optionally at least about 1.1, optionally at least about 1.2, optionally at least about 1.3, optionally at least about 1.4, optionally at least about 1.5, optionally at least about 1.6, optionally at least about 1.7, optionally at least about 1.8, optionally at least about 1.9, optionally at least about 2.0, optionally at least about 2.1, optionally at least about 2.2, optionally at least about 2.3, optionally at least about 2.4, optionally at least about 2.5, optionally at least about 2.6, optionally at least about 2.7, optionally at least about 2.8, optionally at least about 2.9, optionally at least about 3.0, optionally at least about 3.1, optionally at least about 3.2, optionally at least about 3.3, optionally at least about 3.4, optionally at least about 3.5, optionally at least about 3.6, optionally at least about 3.7, optionally at least about 3.8, optionally at least about 3.9, optionally at least about 4.0, optionally at least about 4.1, optionally at least about 4.2, optionally at least about 4.3, optionally at least about 4.4, optionally at least about 4.5, optionally at least about 4.6, optionally at least about 4.7, optionally at least about 4.8, optionally at least about 4.9, and optionally at least about 5.0.

According to some aspects, the chemical agent may be present in the irrigation solution in a concentration sufficient to provide an acceptable log reduction of microbes in a certain time period as described herein. According to some aspects, the chemical agent may be present in the irrigation solution at a concentration of between about 0.001 and 10% w/v, optionally between about 0.001 and 7.5% w/v, optionally between about 0.001 and 5% w/v, optionally between about 0.001 and 2.5% w/v, optionally between about 0.001 and 1% w/v, optionally between about 0.001 and 1% w/v, optionally between about 0.001 and 0.1% w/v, optionally between about 0.001 and 0.01 w/v, optionally between about 0.01 and 10% w/v, optionally between about 0.01 and 7.5% w/v, optionally between about 0.01 and 5% w/v, optionally between about 0.01 and 2.5% w/v, optionally between about 0.01 and 2% w/v, optionally between about 0.01 and 1.5% w/v, optionally between about 0.01 and 1% w/v, and optionally about 0.5% w/v.

According to some aspects, the chemical agent may be present in the irrigation solution at a concentration of between about 0.1 and 0.9% w/v, optionally between about 0.2 and 0.8% w/v, optionally between about 0.3 and 0.7% w/v, and optionally between about 0.4 and 0.6% w/v.

According to some aspects, the chemical agent may be present in the irrigation solution at a concentration of between about 0.1 and 1% w/v, optionally between about 0.2 and 1% w/v, optionally between about 0.3 and 1% w/v, and optionally between about 0.4 and 1% w/v.

It should be understood that according to some aspects, the lavage fluid is not necessarily an irrigation solution as described herein and may be any medically acceptable fluid configured to perform a lavage process as described herein. In one non-limiting example, the lavage fluid may comprise a saline solution. The saline solution may comprise water and sodium chloride in a medically acceptable concentration, such as between about 0.1 and 1%, w/v, optionally about 0.45% w/v, and optionally about 0.9% w/v.

In a preferred embodiment, the lavage fluid may comprise a formulation. The formulation may comprise sodium citrate, citric acid, and sodium lauryl sulfate. The formulation may comprise sodium citrate in a medically acceptable concentration, such as between about 10 to 50 g/l, optionally between about 20 to 40 g/l, and optionally about 30 g/l. The formulation may comprise citric acid in a medically acceptable concentration, such as between about 10 to 50 g/l, optionally between about 20 to 40 g/l, and optionally about 32 g/l. The formulation may comprise sodium lauryl sulfate in a medically acceptable concentration, such as between about 0.1 to 5 g/l, optionally between about 0.5 to 2 g/l, and optionally about 1 g/l.

In a preferred embodiment, the lavage fluid may comprise a formulation. The formulation may comprise sodium acetate, acetic acid, benzalkonium chloride, and ethanol. The formulation may comprise sodium acetate in a medically acceptable concentration, such as between about 10 to 50 g/l, optionally between about 20 to 40 g/l, and optionally about 30 g/l. The formulation may comprise acetic acid in a medically acceptable concentration, such as between about 20 to 100 g/l, optionally between about 40 to 80 g/l, and optionally about 50 g/l. The formulation may comprise benzalkonium chloride in a medically acceptable concentration, such as between about 0.1 to 5 g/l, optionally between about 0.5 to 2 g/l, and optionally about 1 g/l. The formulation may comprise ethanol in a medically acceptable concentration, such as between about 50 to 150 g/l, optionally between about 75 to 125 g/l, and optionally about 100 g/l.

According to some aspects, the lavage fluid, such as an irrigation solution as described herein, may comprise a visualizing aid. As used herein, the term “visualizing aid” refers to a component in a lavage fluid configured to aid in visualizing the application of the lavage fluid. Example visualizing agents include, but are not limited to, tinting agents, staining agents, and radiopaque agents. It should be understood that the visualizing agent may be the same as or different from one of the other components of the lavage fluid. For example, the irrigation agent may function as a visualizing agent. Additionally or alternatively, the lavage fluid may comprise a visualizing agent that is disparate from the chemical agent.

According to some aspects, the lavage fluid may comprise a tinting agent. As used herein, the term “tinting agent” refers to a component sufficient to provide an observable color to a fluid. The tinting agent may be sufficient to allow visualization of the lavage fluid upon application to a surface. In some non-limiting examples, the tinting agent may comprise an anionic tinting agent, such as an anionic dye. The anionic dye may be any dye suitable for medical use, such as dyes approved by the Food and Drug Administration for use in food, drugs, and/or cosmetics (i.e., “D&C” or “FD&C” dyes). Example anionic dyes include, but are not limited to, FD&C Blue No. 1 (Brilliant Blue FCF), FD&C Blue No. 2 (Indigo Carmine), FD&C Green No. 3 (Fast Green FCF), FD&C Red No. 3 (Erythrosine), FD&C Red No. 40 (Allura Red), FD&C Yellow No. 5 (Tartrazine), FD&C Yellow No. 6 (Sunset Yellow FCF), D&C Yellow No. 8 (Fluorescein), D&C Orange No. 4, and combinations thereof. Combinations may be implemented to arrive at a particular color. For example, an orange tint may comprise both FD&C Red No. 40 and D&C Yellow No. 8. Additionally or alternatively, the tinting agent may comprise a chemical compound that is observable upon exposure to visible and/or non-visible light, including, but not limited to, vitamin B-12, medical honey, fluorescent polymeric nanoparticles, water soluble luminescent carbon nanodots, quinine, and combinations thereof.

According to some aspects, the lavage fluid, such as an irrigation solution as described herein, may comprise a staining agent. As used herein, the term “staining agent” refers to a component sufficient to temporarily or permanently color a surface with which it comes in contact.

According to some aspects, the lavage fluid, such as an irrigation solution as described herein, may comprise a radiopaque agent. As used herein, the term “radiopaque agent” refers to a component that is opaque to the radio wave and x-ray portion of the electromagnetic spectrum sufficient for visualization. In some non-limiting examples, the radiopaque agent may comprise barium, iodine, iron oxide nanoparticles, gadolinium complex nanospheres, silica nanospheres, and combinations thereof.

According to some aspects, the lavage fluid, such as an irrigation solution as described herein, may be basic, neutral, or acidic. According to some aspects, the lavage fluid may have a pH of between about 1 and 10, optionally between about 1 and 7, optionally between about 1 and 6, and optionally between about 2 and 5.5.

According to some aspects, the lavage fluid, such as an irrigation solution as described herein, may comprise a buffer system. As used herein, the term “buffer system” refers to a component present in a composition or solution which may provide a resistance to significant change in pH caused by a strong acid or base. A buffer system may comprise a single agent or more than one agent, such as a weak acid and its conjugate base. A buffer system may provide a resistance to a significant pH change by interacting with a strong acid or strong base in a composition or solution, thereby at least partially preventing the pH of the composition or solution from changing significantly.

Generally, a buffer system has one or more buffer ranges wherein the buffer system has the ability to provide resistance to significant pH change. When a composition or solution comprising the buffer system has a pH inside the buffer system's buffer range, the pH of the composition or solution will not change significantly with the addition of equimolar amounts of a strong acid or strong base.

The buffer range of a buffer system is related to the acid dissociation constant (Kₐ) of one or more weak acids comprised by the buffer system. The term “acid dissociation constant” refers to the equilibrium constant of a dissociation reaction of an acid. The midpoint of a buffer range for a buffer system is generally about the logarithmic measure of the acid dissociation constant (i.e., the pKa, equal to −log₁₀ Kₐ) of a weak acid comprised by the buffer system.

According to some aspects, the lavage fluid, such as an irrigation solution as described herein, may comprise a stabilizing agent. As used herein, the term “stabilizing agent” refers to any component that supports the stability of a lavage fluid not otherwise explicitly described herein.

According to some aspects, the lavage fluid may be the lavage fluid as described in U.S. application Ser. No. 17/152,565, hereby incorporated by reference in its entirety.

The system according to the present disclosure comprises a sealed body configured to contain a lavage fluid as described herein. According to some aspects, the sealed body may be compressible. As used herein, the term “compressible” is used in its ordinary and common way, and refers to a reduction in volume to create a pressure differential to support optimal fluid flow. In some aspects, this may include the ability to reversibly reduce in volume without unacceptable changes, such as an unacceptable permanent change to size, to shape, and/or to one or more of the properties as described herein. According to some aspects, the sealed body may be configured such that upon compression, at least a portion of the lavage fluid contained therein is dispensed. It should be understood that as used herein, “dispense” (alternatively referred to as “discharge”) may refer to transferring the lavage fluid to a surface.

According to some aspects, the sealed body may be collapsible. As used herein, the term “collapsible” is used in its ordinary and common way, and refers to the ability to permanently reduce in volume. For example, a collapsible body as described herein may have a first volume when a first volume of fluid is contained therein. When at least a portion of the fluid is dispensed, the collapsible body may collapse to have a second volume, the second volume being less than the first volume. It should be understood that a collapsible body will advantageously reduce the volume of waste (e.g., the volume of the body after the fluid therein has been dispensed). A collapsible body may further provide for a more efficient fluid discharge.

According to some aspects, the sealed body may be configured to allow at least a 10% reduction in volume when compressed and/or collapsed, optionally at least a 20% reduction in volume, optionally at least a 30% reduction in volume, optionally at least a 40% reduction in volume, optionally at least a 50% reduction in volume, optionally at least a 60% reduction in volume, optionally at least a 70% reduction in volume, optionally at least a 80% reduction in volume, and optionally at least a 90% reduction in volume.

According to some aspects, the scaled body may comprise a body material that is compatible with the lavage fluid contained therein, that is, a material that does not chemically or physically react with the lavage fluid or otherwise render the lavage fluid unfit for medical use.

According to some aspects, the body material may be sufficient to prevent unacceptable vapor or chemical loss from a lavage fluid contained therein over a certain period of shelf life. It should be understood that “unacceptable vapor or chemical loss” may be a loss that results in the lavage fluid becoming unsuitable for its intended use. Vapor or chemical loss may result from, for example, adsorption or absorption of the chemical by a material (e.g., by the body material), evaporation of solution, evaporation of a component of a solution (e.g., an antiseptic agent of an antiseptic solution), or a combination thereof. In one non-limiting example wherein the lavage fluid comprises water and iodine as described herein, the body material may be sufficient to prevent water vapor loss and/or iodine loss over a certain period of shelf life.

As used throughout this application, the term “shelf life” refers to the length of time that a product (e.g., an antiseptic solution) may be stored while remaining within the specifications required for the form, fit, and function of the product. Shelf life may be determined by measuring certain characteristics of the product that may indicate that the product is unfit for medical use. For example, shelf life may be determined by measuring the concentration of impurities in the product, the color change of the product, the concentration of insoluble particles in the product, the potency of an active agent contained by the product (e.g., an antiseptic agent), the concentration of one or more components of the product, the pH of the product, and/or the sterility of the product after storage in long-term storage conditions. As used herein, the term “long-term storage conditions” refers to environmental conditions sufficient for a product to be acceptably stored for more than 72 hours. According to some aspects, long-term storage conditions may refer to a temperature of about 25° C., and a relative humidity of about 60%. Additionally or alternatively, shelf life may be determined by measuring the concentration of impurities in the product, the color change of the product, the concentration of insoluble particles in the product, the potency of an active agent of the product, the concentration of one or more components of the product, the pH of the product, and/or the sterility of the product after storage at 37° C., and 65% relative humidity. Additionally or alternatively, shelf life may be determined by measuring the concentration of impurities in the product, the color change of the product, the concentration of insoluble particles in the product, the potency of an active agent of the product, the concentration of one or more components of the product, the pH of the product, and/or the sterility of the product after storage at between about 15 and 30° C. with excursions at a temperature of no more than about 40° C.

According to some aspects, the period of shelf life may be at least about 20 months, optionally at least about 21 months, optionally at least about 22 months, optionally at least about 23 months, optionally at least about 24 months, optionally at least about 25 months, optionally at least about 26 months, optionally at least about 27 months, optionally at least about 28 months, optionally at least about 29 months, optionally at least about 30 months, optionally at least about 31 months, optionally at least about 32 months, optionally at least about 33 months, optionally at least about 34 months, optionally at least about 35 months, optionally at least about 36 months, optionally at least about 37 months, optionally at least about 38 months, optionally at least about 39 months, and optionally at least about 40 months.

According to some aspects, the body material may be sufficient for sterilization by any known sterilization techniques useful according to the present disclosure, including moist heat sterilization (i.e., autoclaving), gas sterilization, gamma irradiation, electron-beam (e-beam) sterilization, aseptic manufacturing processes (e.g., aseptic filtration and/or blow-fill-seal operations), and combinations thereof. Preferably, the body material may be sufficient for sterilization by gamma irradiation, wherein the body material is compatible to the process of gamma irradiation. According to some aspects, the body material does not chemically or physically react with the lavage fluid or otherwise render the lavage fluid unfit for medical use due to the gamma irradiation process. According to some aspects, a body material may be determined to be sufficient for sterilization if a container comprising the body material has a Sterility Assurance Level (SAL) of at least 106 after sterilization and provides an acceptable result upon integrity testing for the container closure after sterilization.

According to some aspects, the body material may have a sufficient mechanical strength such that the body provides an acceptable response to impact, vibration, shaking, or a combination thereof. According to some aspects, an acceptable response refers to a response compliant with ASTM D4169-16 (Standard Practice for Performance Testing of Shipping Containers and Systems). ASTM D4728-06 (Standard Test Method for Random Vibration Testing of Shipping Containers), ASTM D642-15 (Standard Test Method for Determining Compressive Resistance of Shipping Containers. Components, and Unit Loads), or any combination thereof. According to some aspects, the body material may be safe for biomedical use. For example, the body material may comply with ISO 10993 and/or with REACH requirements. According to some aspects, the body material may be sufficient to exhibit at least a portion of the characteristics described herein over a certain period of the lavage fluid's shelf life at a temperature of between about 15 and 30° C. with excursions at a temperature of no more than about 40° C. Additionally or alternatively, the body material may be sufficient to exhibit at least a portion of the characteristics described herein over a certain period of the lavage fluid's shelf life after storage at about 25° C., and 60% relative humidity. Additionally or alternatively, the body material may be sufficient to exhibit at least a portion of the characteristics described herein over a certain period of the lavage fluid's shelf life after storage at about 37° C., and 65% relative humidity.

The body material may be rigid or flexible. As used herein, the term “rigid” refers to a stiffness sufficient to resist deformation upon normal operating forces. As used herein, the term “flexible” refers to the ability to bend or compress under normal operating forces.

Example body materials include, but are not limited to, glass, plastic, paper, foil, and any combination thereof. Example plastics useful according to the present disclosure include, but are not limited to, high-density polyethylene (HDPE), low-density polyethylene (LDPE), polypropylene, polystyrene, nylon, and any combination thereof. According to some aspects, the body material may be a lined and/or coated material, such as a lined and/or coated paper.

According to some aspects, the body material may be polypropylene. Preferably, the body material may be a radiation grade polypropylene. Herein, if the body material is a radiation grade polypropylene, the body material may be able to undergo terminal sterilization. The body material according to the present disclosure may be configured to undergo gamma irradiation as part of terminal sterilization. A radiation grade polypropylene plastic as the body material allows the sealed body containing the lavage fluid to undergo gamma irradiation wherein the sterilization takes place with the lavage fluid already present inside the sealed body.

The sealed body according to the present disclosure, upon undergoing gamma irradiation may maintain flexibility, wherein the lavage fluid contained therein is not significantly affected by the gamma irradiation, that is, the stability and integrity of the lavage fluid may be maintained. Herein, the sealed body maintains its ability to bend or compress under normal operating forces. The sealed body according to the present disclosure, upon undergoing gamma irradiation may not be significantly affected by the gamma irradiation, wherein the stability and integrity of the sealed body may be maintained.

According to some aspects, the scaled body, upon undergoing gamma radiation may undergo certain material characteristic changes that may help in reducing the stretching of the sealed body when the flow-adjusting member creates a disruption in the sealed body, as will be described herein. The reduction of stretching of the sealed body allows the flow-adjusting member to easily create a disruption in the sealed body, as will be described herein.

According to some aspects, the system comprises a flow-adjusting member which is configured to create a disruption in the sealed body to facilitate the discharge of the lavage fluid contained therein, in some embodiments, the flow-adjusting member creates a cut in the sealed body to facilitate the discharge of the lavage fluid. In some embodiments, the flow-adjusting member creates a cut in a wall of the sealed body.

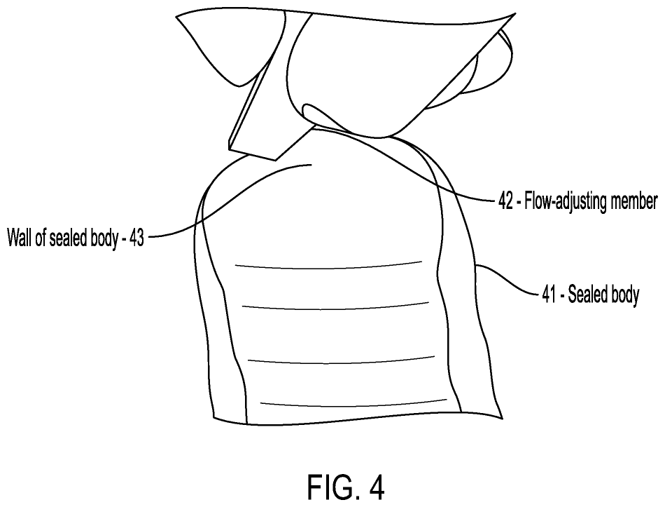

According to some aspects, the sealed body containing the lavage fluid may have variations in the thickness of walls of the sealed body. In particular, one or more wall portions of the sealed body may be of a different thickness as compared to the thickness of other portions or walls of the sealed body. Preferably, the thickness of a certain wall portion may be lesser as compared to other portions or walls of the sealed body. The thickness of such certain wall portion may be at most 95% of the thickness of other portions or walls, optionally at most 90%, optionally at most 85%, optionally at most 80%, optionally at most 75%, optionally at most 70%, optionally at most 65%, optionally at most 60%, optionally at most 55%, optionally at most 50%, optionally at most 45%, optionally at most 40%, optionally at most 35%, optionally at most 30%, and, optionally at most 25% of the thickness of other portions or walls. The wall portion having lesser thickness as described above may be located on the top, bottom or side walls of the sealed body. Preferably, the wall portion having lesser thickness as described above may be the bottom wall of the sealed body in relation to the ground. As seen in FIG. 4, the reduction of thickness in the wall portion 43 allows the flow-adjusting member 42 external to the scaled body 41 to easily create a disruption (such as a cut) in the sealed body. In a related embodiment, the variation of wall thicknesses as well as the trapped gas within the sealed body cooperate in venting the sealed body by allowing the flow-adjusting member to create a disruption in the sealed body. Preferably, the trapped gas is under pressure that is higher than atmospheric pressure. The trapped gas may comprise air and/or an inert gas such as nitrogen.

According to some aspects, the sealed body may be self-supported. According to some aspects, one of the walls of the sealed body is configured to be laid on a surface in a stable manner. Herein, one of the walls of the sealed body supports the rest of the sealed body when laid on a surface to provide a stable position for an individual to use the flow-adjusting member to create a disruption in the sealed body to facilitate the discharge of the lavage fluid. For example, an individual can easily place the sealed body on a table and stably create a cut with the flow-adjusting member.

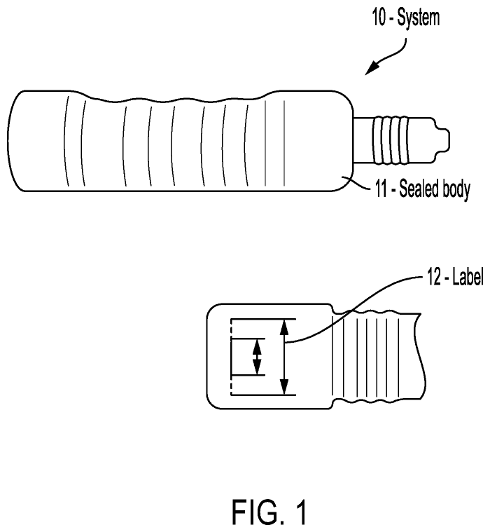

According to some aspects, as seen in FIG. 1, the sealed body 11 may be labelled 12 to indicate the location on the sealed body where it is configured to be disrupted by the flow-adjusting member, e.g., by the individual using the flow-adjusting member to create a disruption in the sealed body. According to some aspects, the sealed body is labelled using the process selected from the group consisting of embossing, engraving, marking, etc.

The sealed body according to the present disclosure is configured to dispense a lavage fluid, such as an irrigation solution, contained therein via one or more mechanisms. According to some aspects, the body may be configured to dispense the lavage fluid upon compression as described herein. For example, the sealed body may be configured to dispense at least a portion of the lavage fluid contained therein in response to compression, such as squeezing. Additionally or alternatively, the sealed body may be configured to dispense at least a portion of the lavage fluid contained therein in response to longitudinal compression.

Additionally or alternatively, the sealed body may be configured to dispense at least a portion of the lavage fluid contained therein upon orienting the body in a certain orientation. Preferably, the flow-adjusting member creates a disruption, such as a cut, puncture, hole, or slit, in the sealed body through which lavage fluid may be dispensed. The sealed body may be configured such that when provided in a certain orientation (e.g., wherein the flow-adjusting member-created disruption is provided at or near the bottom of the sealed body in relation to the ground), at least a portion of the lavage fluid is dispensed by the force of gravity.

The sealed body may further comprise a positioning component that allows the sealed body to be arranged in a certain orientation. The positioning component may be any component configured to position and/or fix the sealed body in a selected orientation, such as a hook, strap, snap, button, tie, or combination thereof. The positioning component may be integral to the scaled body and/or may be a separate component configured to interact with the body, such as a strap attachable to the body. The positioning component may be configured to interact with a second positioning component, such as an extension arm configured to interact with a hook comprised by and/or attached to the sealed body.

It should be understood that the sealed body may be configured to dispense the lavage fluid via one or a combination of the mechanisms as described herein. For example, the sealed body may be configured to dispense the lavage fluid upon compression in conjunction with the force of gravity. According to some aspects, the sealed body may be configured to selectably dispense the lavage fluid via one or more of the mechanisms as described herein. In one non-limiting example, the sealed body may be configured to dispense the lavage fluid upon compression both with and without any external force. In this way, the user may select a desired delivery mechanism based on physical limitations (e.g., the physical capabilities of the user), a desired fluid flow force, a desired fluid flow rate, a desired fluid flow design (e.g., pulsed or constant), or a combination thereof.

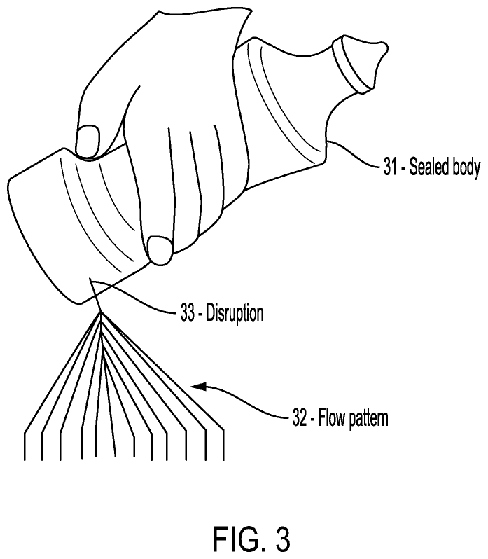

According to some aspects, the flow-adjusting member creates a disruption in the scaled body to facilitate discharge of the lavage fluid through the disruption. Herein, the lavage fluid is discharged in a particular flow pattern. According to some aspects, as seen in FIG. 3, the shape and size of the disruption 33 created by the flow-adjusting member determines the flow pattern 32 of the lavage fluid from the sealed body 31.

According to some aspects, the flow pattern of the lavage fluid is described as the lavage fluid being discharged at a particular pressure. According to some aspects, the lavage fluid is discharged from the sealed body at a pressure under 15 psi, under 12 psi, under 10 psi, under 8 psi, or under 5 psi.

According to some aspects, the flow pattern of the lavage fluid is described as the lavage fluid being discharged in a spray wave of a certain area. According to some aspects, the lavage fluid is discharged from the sealed body in a spray wave of 10 square inches, 9 square inches, 8 square inches, 7 square inches, 6 square inches, 5 square inches, 4 square inches, 3 square inches, 2 square inches, or 1 square inch. Particularly, the spray wave may be in any shape.

According to some aspects, the flow pattern of the lavage fluid is described as substantially all of the lavage fluid being discharged from the sealed body within 20 seconds, less than 20 seconds, within 15 seconds, less than 15 seconds, within 10 seconds, less than 10 seconds, or within 5 seconds.

According to some aspects, the sealed body may be configured to dispense at least about 75% of the lavage fluid contained therein, optionally at least about 80%, optionally at least about 85%, optionally at least about 90%, optionally at least about 95%, and optionally about 100%. The sealed body may be configured to continually dispense the lavage fluid and/or to intermittently dispense the lavage fluid. In one non-limiting example, the sealed body may be configured to intermittently dispense the lavage fluid such that the lavage fluid is only dispensed upon compression of the sealed body.

The sealed body may be configured to contain a volume of lavage fluid sufficient to perform at least a portion of a lavage process. According to some aspects, the sealed body may be configured to contain between about 250 and 2000 mL of fluid, and optionally between about 500 and 1000 mL. According to some aspects, the sealed body may be configured to contain about 500 mL of fluid. According to some aspects, the sealed body may be configured to contain about 1 L of fluid.

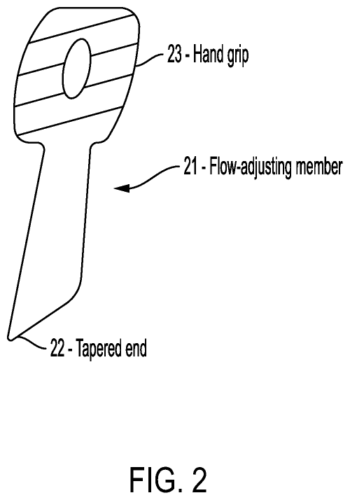

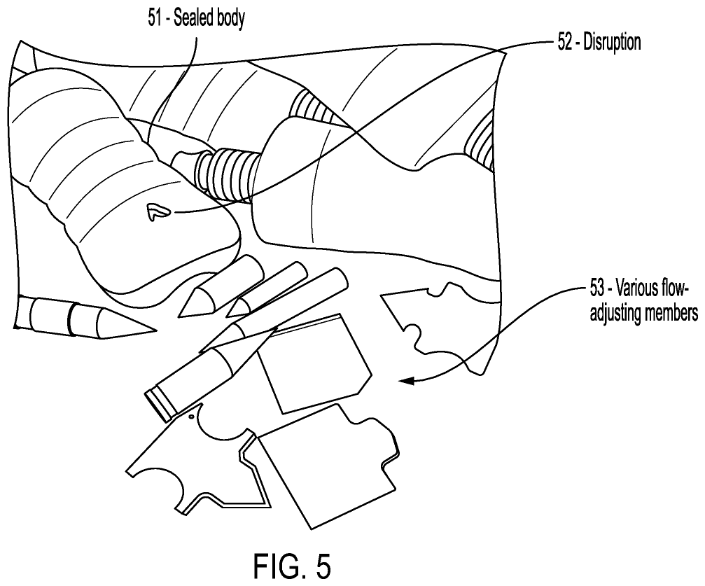

The system according to the present disclosure, comprises a flow-adjusting member configured to create a disruption in the sealed body to facilitate the discharge of the lavage fluid. According to some aspects, as seen in FIG. 2, the flow-adjusting member 21 comprises a tool which is provided with the sealed body as a system, to create a disruption in the sealed body. The tool 21 preferably comprises a tapered end 22 which creates the disruption in the sealed body. A non-limiting example of the tool is a tool with a tapered end which can be used to create a disruption, such as a cut, puncture, hole, or slit, or a tool which can pierce or spike the sealed body at the selected location. The tool may be selected from the group consisting of a generally cylindrically-shaped tool with a tapered end, a knife, a screw, a pin, a blade, a tapered rod, spike, or any tool with a tapered first end 22 and a hand grip 23 towards the second end to create the disruption in the sealed body. As seen in FIG. 5, a variety of flow-adjusting members 53 may be used to create a disruption 52 in the sealed body 51.

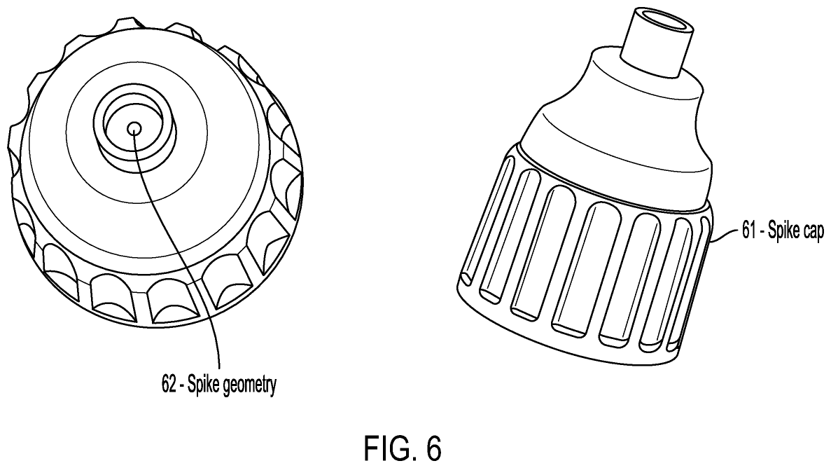

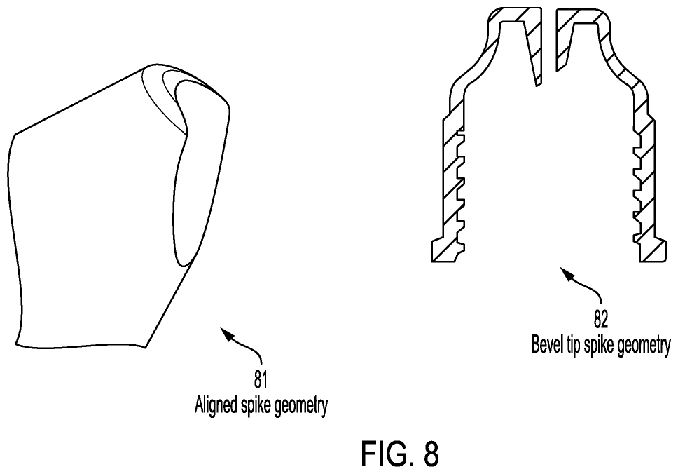

According to some aspects, as seen in FIG. 6, the flow-adjusting member comprises a spike cap 61. The spike cap is configured to be attached to an end of the sealed body to facilitate the discharge of the lavage fluid contained in the sealed body. Herein, the spike of the spike cap creates a disruption on the sealed body when the spike cap is attached to the sealed body to ensure that the lavage fluid is discharged through that cut. According to some aspects, the spike cap comprises a spike geometry 62. The spike geometry determines the shape of the spike which is to create the disruption in the sealed body. According to some aspects, as seen in FIG. 8, the spike geometry comprises a bevel tip 82 or an aligned tip 81. Herein, the spike geometry determines the flow pattern of the lavage fluid being discharged from the sealed body, as described herein. The spike geometry also determines the force needed by the individual spiking the sealed body to spike the sealed body by attaching the spike cap to the sealed body to facilitate the lavage fluid discharge.

According to some aspects, the sealed body may further comprise buttress threads on a first end of the sealed body. According to some aspects, the spike cap may further comprise buttress threads on a first end of the spike cap. Herein, the buttress threads comprising ends of the sealed body and the spike cap are attached to each other, or screwed, or threaded together to ensure the spike cap creates a cut in the sealed body for the discharge of the lavage fluid.

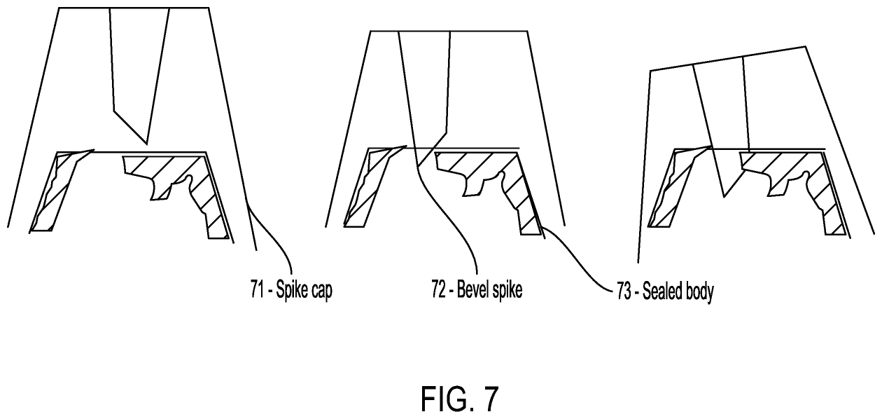

According to some aspects, as seen in FIG. 7, the spike cap 71 with a bevel tip 72 facilitates the process of creating a disruption in the scaled body 73 when the spike cap is attached to the sealed body, wherein the bevel tip pierces the selected location on the sealed body. According to some aspects, the spike cap with an aligned tip aligns the piercing process with the threading process when the spike cap is attached to the sealed body.

According to some aspects, more than one spike cap is provided with the sealed body. According to some aspects, spike caps with different spike geometries are provided with the scaled body to achieve different flow patterns of the lavage fluid, as desired.

According to some aspects, the flow-adjusting member is integral to the system comprising the sealed body. According to some aspects, the flow-adjusting member may or may not be in fluid communication with the sealed body during the discharge of the lavage fluid from the sealed body.

According to some aspects, the flow-adjusting member comprises a particular material, wherein the material has the ability to utilize sharp edges on itself, thus eliminating metal sharps which create risks within a healthcare environment. According to some aspects, the flow-adjusting member is made of plastic material. Particularly, the flow-adjusting member may be made of a plastic including, but not limited to, high-density polyethylene (HDPE), low-density polyethylene (LDPE), polypropylene, polystyrene, acrylic, nylon, polycarbonate, polymethyl methacrylate (PMMA), polyacrylonitrile (PAN), polyester, acrylonitrile butadiene styrene (ABS), polyvinyl chloride (PVC), and polyvinylidene fluoride (PVDF).

According to some aspects, the flow-adjusting member may be made of metal material or an alloy material. Particularly, the flow-adjusting member may be made of a metal or alloy including, but not limited to, stainless steel, cobalt-chrome alloy, titanium, nickel-titanium alloy, gold, platinum, silver, iridium, tantalum, tungsten, aluminum, copper, and magnesium.

According to some aspects, the sealed body may be provided with a removable lid to prevent fluid discharge from the sealed body, for example, during storage or transportation of the sealed body.

According to some aspects, the system may be configured to provide an acceptable fluid flow rate for a lavage process. As used herein, the term “fluid flow rate” refers to the rate at which a fluid is applied to a surface, such as to a human subject during a lavage process. The fluid flow rate may depend at least partially on the delivery mechanism and/or the properties of the flow-adjusting member as described herein. According to some aspects, a fluid flow rate may be related to a fluid flow force. For example, an increased fluid flow rate may correspond with an increased fluid flow force, and vice versa. The system according to the present disclosure may be configured to provide different, selectable fluid flow rates.

According to some aspects, the system may be configured to provide an acceptable fluid flow force for a lavage process. As used herein, the term “fluid flow force” refers to the force of a fluid acting on a surface, such as on a human subject during a lavage process. An acceptable fluid flow force may be determined based on the lavage process requirements.

It should be understood that the fluid flow force provided by the systems as described herein may depend at least partially on the delivery mechanism and/or the properties of the flow-adjusting member as described herein. The system according to the present disclosure may be configured to provide different, selectable fluid flow forces. It should be understood that each of the selectable fluid flow forces may correspond with, for example, a specific delivery mechanism, a specific flow-adjusting member, or a combination thereof, as described herein.

According to some aspects, the system is configured to provide an acceptable fluid flow design for a lavage process. As used herein, the term “fluid flow design” refers to the design with which a fluid is dispensed from a device and/or applied to a surface, such as to a human subject during a lavage process. In some non-limiting examples, the fluid flow design may comprise a fluid mist (i.e., a suspension of finely divided fluid in a gas), a fluid stream (i.e., a steady succession of fluid), a fluid spray (i.e., finely divided fluid), or a combination thereof. The fluid flow design may be constant (e.g., fluid continually dispensed from a device and/or applied to a surface) or pulsed (e.g., the fluid intermittently dispensed from a device and/or applied to a surface).

A fluid flow design may additionally or alternatively refer to the angle at which a fluid flow path is dispensed from a device and/or applied to a surface. For example, a fluid flow path may have a fluid flow design that is about perpendicular to a longitudinal axis of a sealed body as described herein.

Additionally or alternatively, a fluid flow design may refer to the geometric shape of a fluid path. It should be understood that the geometric shape of a fluid path refers to a shape defined by the cross-sectional view of a fluid flow path in any of the x-direction, y-direction, and z-direction.

It should be understood that the fluid flow design may depend at least in part on the delivery mechanism and/or the flow-adjusting member as described herein.

According to some aspects, one or more components of the system described herein may be provided in sterile packaging. As used herein, the term “sterile packaging” refers to packaging that provides a sterile environment so as to maintain sterility of a contained sterile product. Example sterile packaging includes, but is not limited to, sterile blister packaging, sterile safe-edge trays, sterile surgical trays, sterile customized thermoforms, sterile bags, sterile plastic formed trays with top lids, and combinations thereof. It should be understood that one or more components of the system may be provided in the same sterile packaging and/or separate sterile packaging from at least one other component of the system. For example, a first component of the system may be contained in a first sterile packaging and a second component of the system may be contained in a second sterile packaging. In one non-limiting example, the system may comprise a scaled body contained in a first sterile packaging and a flow-adjusting member contained in a second sterile packaging. It should be understood that providing one or more components of the system in different sterile packaging allows for the removal of each component of the system immediately prior to its use, thus preventing one or more components from prolonged exposure to an unsterile environment. In this way, a fully assembled sterile presentation of the system may be achieved.

While the aspects described herein have been described in conjunction with the example aspects outlined above, various alternatives, modifications, variations, improvements, and/or substantial equivalents, whether known or that are or may be presently unforeseen, may become apparent to those having at least ordinary skill in the art. Accordingly, the example aspects, as set forth above, are intended to be illustrative, not limiting. Various changes may be made without departing from the spirit and scope of the disclosure. Therefore, the disclosure is intended to embrace all known or later-developed alternatives, modifications, variations, improvements, and/or substantial equivalents.

Thus, the claims are not intended to be limited to the aspects shown herein, but are to be accorded the full scope consistent with the language of the claims, wherein reference to an element in the singular is not intended to mean “one and only one” unless specifically so stated, but rather “one or more.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

Further, the word “example” is used herein to mean “serving as an example, instance, or illustration.” Any aspect described herein as “example” is not necessarily to be construed as preferred or advantageous over other aspects. Unless specifically stated otherwise, the term “some” refers to one or more. Combinations such as “at least one of A, B, or C,” “at least one of A, B, and C,” and “A, B, C, or any combination thereof” include any combination of A, B, and/or C, and may include multiples of A, multiples of B, or multiples of C. Specifically, combinations such as “at least one of A, B, or C,” “at least one of A, B, and C,” and “A, B, C, or any combination thereof” may be A only, B only, C only, A and B, A and C, B and C, or A and B and C, where any such combinations may contain one or more member or members of A, B, or C. Nothing disclosed herein is intended to be dedicated to the public regardless of whether such disclosure is explicitly recited in the claims.

The word “about” is used herein to mean within +5% of the stated value, optionally within +4%, optionally within +3%, optionally within +2%, optionally within +1%, optionally within +0.5%, optionally within +0.1%, and optionally within +0.01%.

What is claimed is:

1. A system for applying a lavage fluid to a surface, the system comprising:
a sealed body containing a lavage fluid, wherein the sealed body and lavage fluid contained therein are terminally-sterilized; and
a flow-adjusting member, wherein the flow-adjusting member is configured to create a disruption in the sealed body to facilitate discharge of the lavage fluid in a flow pattern.

2. The system of claim 1, wherein the flow-adjusting member comprises a tool with a tapered end.

3. The system of claim 1, wherein the flow-adjusting member comprises a spike cap configured to be attached to a first end of the sealed body, wherein the spike cap comprises a spike geometry.

4. The system of claim 3, wherein the body comprises buttress threads on the first end of the sealed body.

5. The system of claim 4, wherein the spike cap further comprises buttress threads which attach to the buttress threads on the first end of the sealed body.

6. The system of claim 3, wherein the spike geometry comprises a bevel tip.

7. The system of claim 3, wherein the spike geometry comprises an aligned tip.

8. The system of claim 1, wherein the flow-adjusting member is separate from the sealed body during the discharge of the lavage fluid.

9. The system of claim 1, wherein the flow-adjusting member is in fluid communication with the sealed body during discharge of the lavage fluid.

10. The system of claim 1, wherein the flow-adjusting member creates a cut in the sealed body, wherein a shape and size of the cut determines the flow pattern of the lavage fluid.

11. The system of claim 1, wherein the sealed body is compressible, and the sealed body is configured such that at least a portion of the lavage fluid is discharged through the disruption, upon compression of the sealed body.

12. The system of claim 1, wherein the sealed body is configured such that at least a portion of the lavage fluid is discharged through the disruption, with or without compression of the sealed body.

13. The system of claim 1, wherein the sealed body is collapsible.

14. The system according to claim 1, wherein the flow-adjusting member is integral to the sealed body.

15. The system of to claim 1, wherein the lavage fluid comprises a chemical agent and water, the chemical agent comprising iodine.

16. The system of claim 1, wherein the flow pattern comprises substantially all of the lavage fluid being discharged from the sealed body within 20 seconds.

17. The system of claim 1, wherein the flow pattern comprises the lavage fluid being discharged at a pressure under 15 psi.

18. The system of claim 1, wherein the flow pattern comprises of the lavage fluid being discharged in a spray wave of 6 square inches.

19. The system of claim 1, wherein the flow-adjusting member comprises a material selected from the group consisting of plastics, metals, and alloys.

20. The system of claim 1, wherein the sealed body comprises a plastic that is compatible with gamma radiation.

21. The system of claim 1, wherein the sealed body and lavage fluid contained therein are terminally-sterilized via gamma radiation.
